(19) United States
(12) Patent Application Publication
(10) Pub. No.: US 2021/0369154 A1
(43) Pub. Date: Dec. 2, 2021

(54) METHOD FOR NONINVASIVE DETERMINATION OF HEMOGLOBIN AND OXYGEN CONCENTRATIONS IN THE BLOOD

(71) Applicant: TELEBIOMET, Limited

(72) Inventors: Edvard Vladimirovich Kryzhanovsky, Saint-Petersburg (RU); Armen Gareginovich Grigoryan, Saint-Petersburg (RU); Vladimir Victorovich Kovalev, Saint-Petersburg (RU); Aleksandr Vladimirovich Chistov, Saint-Petersburg (RU)

(73) Assignee: TELEBIOMET, Limited, Moscow (RU)

(21) Appl. No.: 16/336,688

(22) PCT Filed: Oct. 2, 2017
(86) PCT No.: PCT/RU2017/000731

§ 371 Date: Aug. 10, 2021

(51) Int. Cl. A61B 5/1455

(52) U.S. Cl. CPC A61B 5/14551; A61B 2562/0238; A61B 2560/02

(57) ABSTRACT

The invention applies to the analysis of the chemical composition of materials and can be used primarily in diagnostic medical equipment for noninvasive determination of hemoglobin and oxygen concentrations contained in the blood.

The method proposes to alternately irradiate the biological tissue with optical radiation of the first, second, and third wavelength ranges, including 700 nm, 880 nm, and 960 nm, respectively, receive the reflected optical radiation, convert it to an electrical signal, determine the concentration of hemoglobin based on the sum of the electrical signals obtained by irradiating with optical radiation of the first and second ranges, which is reduced by a value determined by the electrical signal obtained upon irradiation with optical radiation of the third range, and determine the concentration of oxygen based on the difference between the electrical signals obtained by irradiating with optical radiation of the second and first ranges, which is reduced by a value determined by the electrical signal obtained by irradiating with optical radiation of the third range.

The invention provides a reduction in the error of determining the concentrations of hemoglobin and oxygen that stems from the presence of water in the biological tissue under study.

### PERTINENT ART

The invention applies to the research and analysis of the chemical composition of materials and can be used primarily in diagnostic medical equipment for non-invasive determination of hemoglobin and oxygen concentrations contained in blood.

### PRIOR ART

Methods and technical means of optical oximetry are most widely used to determine noninvasively oxygen saturation of the blood and the concentration of hemoglobin. These methods utilize the differences that forms of hemoglobin containing and not containing oxygen exhibit in the absorption of optical radiation, as deoxyhemoglobin and oxyhemoglobin significantly absorb red and infrared optical radiation, respectively.

A method for determining the concentration of blood components is known (RU 2344752 C1, 2009), which estimates the hemoglobin concentration by exposing the biological tissue to alternating radiation within visible part of the spectrum with a wavelength of, for example. 590 nm and 650 nm, and further detecting the radiation transferred through the tissue, converting it into an electrical signal, and determining the concentration of hemoglobin in the blood using the amplitude values of the received electrical signals.

Methods for noninvasive determination of oxygen saturation and hemoglobin concentration are implemented in known pulse oximeters (RU 2175523 C1, 2001; RU 2221485 C2, 2004; RU 2233620 C1, 2004; RU 2259161 C1, 2005; RU 2332165 C2, 2008; RU 2496418 C1, 2013). They all expose the biological tissue to alternating red and near infrared optical radiation with various wavelengths, detect the radiation transferred through the tissue, convert it into an electrical signal, and determine the concentration of hemoglobin and oxygen saturation of the blood using the amplitude values of the received electrical signals.

However, all the known methods allow the diagnosis of blood oxygenation only for the parts of biological tissue which are transparent to the optical radiation of these wavelength ranges, which makes it possible to use them only for such relatively thin biological tissues as the finger and earlap.

A single-use pulse oximeter (RU 2428112 C2, 2011) exposes the biological tissue to alternating red and near infrared optical radiation, detects the red and near infrared optical radiation diffusely reflected by the tissue, converts it into an electrical signal, and determines the concentration of hemoglobin and oxygen saturation in the blood using the amplitude values of the received electrical signals.

The use of optical radiation diffusely reflected by the biological tissue in this known method significantly expands its utility, since it is applicable not only for the fingers or earlaps, but also for other biological tissues of the human body, in particular, for the soft tissues of the forehead, frontal bones, and frontal fractions of the brain.

The proposed method for noninvasive determination of the hemoglobin and oxygen concentrations in the blood is most close to an optical method for estimation of blood oxygenation (RU 2040912 C1, 1995), which exposes the biological tissue to alternating red and near infrared optical radiation, detects the red and near infrared optical radiation diffusely reflected by the tissue, converts it into an electrical signal, and determines the concentration of hemoglobin in the blood and oxygen saturation using the amplitude values of the received electrical signals.

All above mentioned methods provide an insufficient accuracy in determining the hemoglobin and oxygen concentrations in the blood. This is due to the measurement error caused by a significant amount of water present in the biological tissue, which has a detectable absorption spectrum within the infrared wavelength bands used in the considered devices

### INVENTION SUMMARY

The proposed invention aims at providing a method for noninvasive determination of hemoglobin and oxygen concentrations in the blood, which increases the measurement accuracy compared to the existing methods.

The proposed invention achieves the aim and the technical result as follows. In accordance with the previously described analogues, the proposed method exposes the biological tissue to alternating red and near infrared optical radiation (in any sequence), detects the optical radiation diffusely reflected by the tissue, converts it into an electrical signal, and determines the concentrations of hemoglobin and oxygen using the received electrical signal. The proposed method differs from the closest analogue by the fact that the biological tissue is exposed to optical radiation of the first wavelength range, including the 700 nm wavelength, to optical radiation of the second wavelength range, including 880 nm, and to optical radiation of the third wavelength range, including 960 nm. The concentration of hemoglobin is determined using the sum of the electrical signals obtained by exposing the biological tissue to optical radiation of the first and second ranges, which Is reduced by a value determined by the electrical signal obtained by exposing the biological tissue to optical radiation of the third range. The concentration of oxygen is determined from the difference between the electrical signals obtained by exposing the biological tissue to optical radiation of the second and first ranges, which is reduced by a value determined by the electrical signal obtained by exposing the biological tissue to optical radiation of the third range.

The hemoglobin concentration in the blood is determined using the experimentally obtained calibration curve for the dependence between the concentration of hemoglobin and the resulting total electrical signal having the value U_{TOT}=U₁+U₂−U₃( [symbol] ₁₃+ [symbol] ₂₃), where U₁, U₂, and U₃ are the values of the electrical signals obtained when the biological tissue is irradiated with optical radiation of the first, second, and third wavelength ranges, respectively; [symbol] ₁₃ and [symbol] ₂₃ are coefficients obtained in advance by processing the known characteristics of the relative spectral sensitivity of the optical receiver and the water absorption spectrum in the first, second, and third wavelength ranges, respectively.

The oxygen concentration in the blood is determined using the experimentally obtained calibration curve for the dependence between the concentration of oxygen and the resulting residual electrical signal having the value U_{DIFF}=U₂−U₁−U₃( [symbol] ₁₃+ [symbol] ₂₃), where U₁, U₂, and U₃ are the values of the electrical signals obtained when the biological tissue is irradiated with optical radiation of the first, second, and third wavelength ranges, respectively, [symbol] ₁₃ and [symbol] ₂₃ are coefficients obtained in advance by processing the known characteristics of the relative spectral sensitivity of the optical receiver and the water absorption spectrum in the first, second, and third wavelength ranges.

The coefficients [symbol] ₁₃ and [symbol] ₂₃ are obtained in advance by processing the known characteristics of the relative spectral sensitivity of the optical receiver and the water absorption spectrum in the first, second, and third wavelength ranges; these coefficients are calculated according to the following expressions: [symbol] ₁₃= [symbol] ₃S₃/ [symbol] ₁/S₁ and [symbol] ₂₃= [symbol] ₃S₃/ [symbol] ₂/S₂, where [symbol] ₁, [symbol] ₂, and [symbol] ₃ are the average values of the water absorption coefficients in the first, second, and third wavelength ranges, respectively; S₁, S₂, and S₃ are the average values of the relative spectral sensitivity of the optical receiver in the first, second, and third wavelength ranges, respectively.

On the one hand, the optical radiation of the first wavelength range, including 700 nm, is much more absorbed by deoxyhemoglobin than oxyhemoglobin. On the other hand, the optical radiation of the second wavelength range, including 880 nm, is more absorbed by oxyhemoglobin than by deoxyhemoglobin. Therefore, the method proposes to expose the biological tissue to optical radiation of the first wavelength range, including 700 nm, and optical radiation of the second wavelength range, including 880 nm, in order to determine the concentration of hemoglobin in the blood from the sum of the electrical signals obtained by irradiating the biological tissues by optical radiation of the first and second ranges, and also to determine the concentration of oxygen in the blood from the difference between the electrical signals obtained by irradiating the biological tissue with optical radiation of the second and first ranges.

At the same time, biological tissues contain a significant amount of water.

Water has the most pronounced absorption spectrum of optical radiation in the wavelength range from 650 nm to 1100 nm with a maximum near the wavelength of 960 nm. Therefore, the presence of water in the biological tissue leads to a distortion of the useful signal, which is manifested in the increase in the electrical signal due to the absorption of optical radiation by the water both in the first wavelength range and, to a much greater extent, in the second wavelength range, introducing a significant error in the determination of the hemoglobin and oxygen concentrations.

To evaluate and exclude the measurement error due to the presence of water in the biological tissue under investigation, the present invention proposes to expose the biological tissue to optical radiation of the third wavelength range, comprising the maximum of the water absorption spectrum at 960 nm, and do it before, after, or between the irradiation with optical radiation of the first wavelength range, including 700 nm, and optical radiation of the second wavelength range, including 880 nm, which provides a useful signal for determining hemoglobin and oxygen concentrations. The optical radiation of the third wavelength range diffusely reflected by the biological tissue is then received and converted to an electrical signal, which mainly depends on the current value of the water concentration in the investigated biological tissue.

Therefore, the determination of the hemoglobin concentration in the blood from the sum of the electrical signals obtained by irradiating the biological tissue with optical radiation of the first and second wavelength ranges, which is reduced by a value determined by the electrical signal obtained by irradiating the biological tissue with optical radiation of the third wavelength range, accounts for the error due to the presence of water in the biological tissue under study, and, thereby, allows to increase the accuracy of determining the hemoglobin concentration.

In addition, the determination of the oxygen concentration from the difference between the electrical signals obtained by irradiating the biological tissue with optical radiation of the second and first wavelength ranges, which is reduced by a value determined by the electrical signal obtained by irradiating the biological tissue with optical radiation of the third wavelength range, also accounts for the error due to the presence of water in the biological tissue, and, thereby, allows to increase the accuracy of determining the oxygen concentration.

The proposed method thus solves the problem and achieves the technical result formulated in the innovation, since the noninvasive determination of hemoglobin and oxygen concentrations in the blood possesses the above-mentioned distinctive features.

### BRIEF DESCRIPTION OF DRAWING

FIG. 1 shows the block diagram of the device that provides the best way to implement the claimed method for noninvasive determination of hemoglobin and oxygen concentrations in the blood, where t is the block of light-emitting diodes (LEDs). 2—the optical receiver, 3—the amplifier, 4—the analog-to-digital converter, 5—the controller, 6—the display unit, and 7 is the biological tissue.

FIG. 2 shows the absorption spectra for oxyhemoglobin, deoxyhemoglobin, and water in the wavelength range from 600 nm to 1100 nm.

### PREFERRED EMBODIMENT OF THE INVENTION

A device that provides the best way to implement the claimed method of noninvasive determination of hemoglobin and oxygen concentrations in blood comprises a series connected optical radiation receiver 2, amplifier 3, analog-digital converter 4, controller 5 and display unit 6, and also an LED unit 1 connected to output of the controller 5.

The LED unit 1 comprises at least one LED configured to emit optical radiation in a first wavelength range (680-720 nm), including 700 nm, for example LED L-132XHT by Kingbright, at least one LED with emission in the second wavelength range (860-900 nm), including 880 nm, for example LED BL-314IR by BetLux, and at least one LED with emission in the third wavelength range (940-980 nm), including 960 nm, for example LED TSUS4400 by Vishay.

A photodiode sensitive to optical radiation in the wavelength range from 570 to 1100 nm, for example photodiode BPW34 by Vishay, is used as the optical radiation receiver 2.

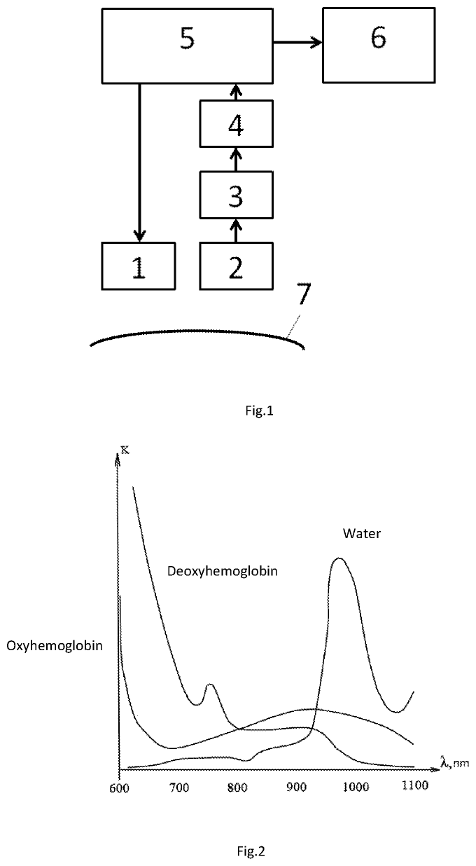

The optical radiation receiver 2 and the LEDs of the LED unit 1 are mounted on a common base (not shown in FIG. 1) that is pressed against the biological tissue 7, and the LEDs are arranged around the optical radiation receiver 2.

A precision operational amplifier, for example AD8604 by Analog Devices, can be used as the amplifier 3.

A high-speed analog-to-digital converter of a large bit width (from 12 bits), for example an analog-to-digital converter AD7655 by Analog Devices, can be used as the analog-to-digital converter 4.

The controller 5 can be any microcontroller having the necessary resources to control an external analog-to-digital converter and sufficient speed, for example ATXmega128A4U by Atmel, equipped with permanent and operative memory devices.

The device that implements the proposed method of noninvasive determination of hemoglobin and oxygen concentrations in the blood works as follows.

To determine the hemoglobin and oxygen concentrations in the blood, the base with the optical radiation receiver 2 and the LEDs of the LED unit 1 is pressed against the biological tissue 7 under study.

When the device is turned on, the LEDs of the LED unit 1 do not emit the optical radiation. The electrical signal from the optical radiation receiver 2. determined by its dark current, is amplified by the amplifier 3 and converted by the analog-to-digital converter 4 into a digital code, which enters the controller 5 and is stored in its operative memory device.

Then, the signals from the controller 5 initiate alternate energy supply of ihe LEDs of the LED unit 1. The sequence in which the LEDs are switched are not important for the proposed method.

For example, when a voltage is applied to a LED of the LED unit 1 that is configured to emit optical radiation in the first wavelength range of 680-720 nm, this LED emits optical radiation of the indicated wavelength range towards the biological tissue 7. A portion of this radiation is absorbed, predominantly by deoxynemogiobin. and a part diffusely reflects and gets to the optical radiation receiver 2, which converts this part of the optical radiation into an electrical signal, determined, to a greater extent, by the concentration of deoxyhemoglobin in the biological tissue 7 and, to a lesser extent, by oxyhemoglobin and water (see FIG. 2). This electrical signal is amplified by the amplifier 3 and. after conversion by the analog-to-digital converter 4 into a digital code, enters the controller 5. In order to account for the measurement error that stems from the dark current of the optical radiation receiver 2. the controller 5 subtracts from the digital code that it has received from the analog-to-digital converter 4 a digital code corresponding to the electric signal from the dark current of the optical radiation receiver 2 (the latter code is stored in the main memory). Then, the controller 5 records this difference, corresponding to the electric signal u₁, to the main memory, and this signal is mainly determined by the concentration of deoxyhemoglobin in the examined biological tissue 7.

Then, the previously turned-on LED turns off, and voltage is applied, for example, to a LED of the LED unit 1 configured to emit optical radiation in the second range with wavelengths of 860-900 nm. This LED emits optical radiation of the indicated wavelength range in the direction of the biological tissue 7. Similarly, the optical radiation receiver 2 converts the diffusely reflected optical radiation into an electrical signal, which is determined predominantly by the oxyhemoglobin concentration in the biological tissue 7 and, to a lesser extent, by deoxyhemoglobin and water (see FIG. 2). This electrical signal is amplified by the amplifier 3 and, after conversion by the analog-to-digital converter 4 into a digital code, enters the controller 5. In order to account for the measurement error that stems from the dark current of the optical radiation receiver 2, the controller 5 subtracts from the digital code that it has received from the analog-to-digital converter 4 a digital code corresponding to the electric signal from the dark current of the optical radiation receiver 2 (the latter code is stored in the main memory). Then, the controller 5 records this difference, corresponding to the electric signal u₂, to the main memory, and this signal is mainly determined by the concentration of oxyhemoglobin in the examined biological tissue 7.

Then, the previously turned-on LED turns off, and voltage is applied to a LED of the LED unit 1 configured to emit optical radiation in the third range with wavelengths of 940-980 nm. This LED emits optical radiation of the indicated wavelength range in the direction of the biological tissue 7. Similarly, the optical radiation receiver 2 converts the diffusely reflected optical radiation into an electrical signal, which is determined predominantly by the concentration of water in the biological tissue 7 and, to a lesser extent, by oxyhemoglobin and deoxyhemoglobin (see FIG. 2). This electrical signal is amplified by the amplifier 3 and, after conversion by the analog-to-digital converter 4 into a digital code, enters the controller 5. In order to account for the measurement error that stems from the dark current of the optical radiation receiver 2, the controller 5 subtracts from the digital code that it has received from the analog-to-digital converter 4 a digital code corresponding to the electric signal from the dark current of the optical radiation receiver 2 (the latter code is stored in the main memory). Then, the controller 5 records this difference, corresponding to the electric signal u₃, to the main memory, and this signal is mainly determined by the concentration of water in the examined biological tissue 7.

Then, the considered processes of sequential switching of the LEDs of the LED unit 1, initiated by the signals from the controller 5, are repeated multiple times, each time followed by conversion of the reflected optical radiation into an electrical signal by the optical radiation receiver 2 and processing of the obtained digital codes by the controller 5. As a result, digital values of the electrical signals u₁, u₂, and u₃ are accumulated in the main memory of the controller 5, which are statistically processed by the controller 5 for filtering the random measurement errors. This processing results in the average numerical values U₁, U₂, and U₃ of the electrical signals u₁, u₂, and u₃. respectively, which are stored in the main memory of the controller 5. Based on the obtained average numerical values U₁, U₂, and U₃ of the electrical signals, the controller 5 calculates the total electric signal according to the following expression:

U_{TOT}=U₁+U₂−

[symbol]

where U₁, U₂, and U₃ are the average numerical values of the electrical signals obtained by exposing the biological tissue 7 to optical radiation of the first, second, and third wavelength ranges, respectively;

[symbol] ₁₃, [symbol] ₂₃ are coefficients obtained in advance by processing the known characteristics of the relative spectral sensitivity of the optical radiation receiver 2 and the water absorption spectrum in the first, second, and third wavelength ranges, respectively, which are stored in the main memory of the controller 5.

Based on the obtained average numerical values U₁, U₂, and U₃ of the electrical signals, the controller 5 calculates the residual electric signal according to the following expression:

U_{DIFF}=U₂−U₁− [symbol] ,

where U₁, U₂, and U₃ are the average numerical values of the electrical signals obtained by exposing the biological tissue 7 to optical radiation of the first, second, and third wavelength ranges, respectively;

[symbol] ₁₃, [symbol] ₂₃ are coefficients obtained in advance by processing the known characteristics of the relative spectral sensitivity of the optical radiation receiver 2 and the water absorption spectrum in the first, second, and third wavelength ranges, respectively, which are stored in the main memory of the controller 5.

The above-mentioned coefficients, which are stored in the main memory of the controller 5, are determined in advance by processing the known characteristics of the relative spectral sensitivity of the optical radiation receiver 2 and the water absorption spectrum in the first, second, and third wavelength ranges, according to the following expressions:

[symbol]

₁₃= [symbol] ₃S₃/ [symbol] ₁/S₁,

[symbol]

₂₃= [symbol] ₃S₃/ [symbol] ₂/S₂,

where [symbol] ₁, [symbol] ₂, and [symbol] ₃ are the average values of the water absorption coefficients in the first, second, and third wavelength ranges, respectively;

S₁, S₂, and S₃ are the average values of the relative spectral sensitivity of the optical radiation receiver 2 in the first, second, and third wavelength ranges, respectively.

The controller 5 determines the concentration of hemoglobin in the blood using the calibration curve between the concentration of hemoglobin and the resulting total electrical signal U_{TOT}. The calibration curve has been experimentally obtained in advance and is stored in the main memory of the controller 5.

The controller 5 determines the concentration of oxygen in the blood using the calibration curve between the concentration of oxygen and the resulting residual electrical signal U_{DIFF}. The calibration curve has been experimentally obtained in advance and is stored in the main memory of the controller 5.

The obtained hemoglobin and oxygen concentrations in the blood are transferred from the controller 5 to the display unit 6, which displays these values to the device operator.

### INDUSTRIAL APPLICABILITY

The authors of the present invention have developed and tested a prototype device that provides the proposed method of noninvasive determination of hemoglobin and oxygen concentrations in the blood The tests of the prototype device showed, firstly, its operability and, secondly, the possibility of achieving the technical result, consisting in increasing the accuracy of determination of hemoglobin and oxygen concentrations by 10-12% reduction of the measurement error due to the presence of water in the biological tissue under study.

1. (canceled)

2. (canceled)

3. (canceled)

4. (canceled)

5. A method for noninvasive determination of the hemoglobin concentration in blood, including:
alternate irradiation of the biological tissue in any sequence with optical radiation of the first wavelength range, including 700 nm, optical radiation of the second wavelength range, including 880 nm, and optical radiation of the third wavelength range, including 960 nm,
reception of the optical radiation diffusely reflected by the biological tissue,
conversion of the received optical radiation into an electrical signal, and
determination of the hemoglobin concentration in blood based on the sum of the electrical signals obtained when the biological tissue is irradiated with optical radiation of the first and second wavelength ranges, which is reduced by a value determined by the electrical signal obtained under irradiation of the biological tissue with optical radiation of the third wavelength range.

6. A method according to claim 5, wherein the hemoglobin concentration in the blood is determined using the experimentally obtained calibration curve between the concentration of hemoglobin and the resulting total electrical signal U_{TOT}=U₁+U₂− [symbol] , where U₁, U₂, and U₃ are the electrical signals obtained by irradiating biological tissue with optical radiation of the first, second, and third wavelength ranges, respectively; [symbol] ₁₃ and [symbol] ₂₃ are coefficients obtained in advance by processing the known characteristics of the relative spectral sensitivity of the optical radiation receiver used in the measurement and the water absorption spectrum in the first, second, and third wavelength ranges, respectively.

7. A method according to claim 6, wherein the described coefficients, obtained by processing the known characteristics of the relative spectral sensitivity of the optical radiation receiver used in the measurement and the water absorption spectrum in the first, second, and third wavelength ranges, are calculated in advance according to the following expressions: [symbol] ₁₃= [symbol] ₃S₃/ [symbol] ₁/S₁ and [symbol] ₂₃= [symbol] ₃S₃/ [symbol] ₂/S₂, where [symbol] ₁, [symbol] ₂, and [symbol] ₃ are the average values of the water absorption coefficients in the first, second, and third wavelength ranges, respectively; S₁, S₂, and S₃ are the average values of the relative spectral sensitivity of the optical radiation receiver in the first, second, and third wavelength ranges, respectively.

8. A method for noninvasive determination of the oxygen concentration in blood, including:
alternate irradiation of the biological tissue in any sequence with optical radiation of the first wavelength range, including 700 nm, optical radiation of the second wavelength range, including 880 nm, and optical radiation of the third wavelength range, including 960 nm,
reception of the optical radiation diffusely reflected by the biological tissue,
conversion of the received optical radiation into an electrical signal, and
determination of the oxygen concentration in blood based on the difference between the electrical signals obtained when the biological tissue is irradiated with optical radiation of the second and first wavelength ranges, which is reduced by a value determined by the electrical signal obtained under irradiation of the biological tissue with optical radiation of the third wavelength range.

9. A method according to claim 8, wherein the oxygen concentration in the blood is determined using the experimentally obtained calibration curve between the concentration of oxygen in the blood and the resulting residual electrical signal U_{DIFF}=U₂−U₁−U₃( [symbol] ₁₃+ [symbol] ₂₃), where U₁, U₂, and U₃ are the electrical signals obtained by irradiating biological tissue with optical radiation of the first, second, and third wavelength ranges, respectively; [symbol] ₁₃ and [symbol] ₂₃ are coefficients obtained in advance by processing the known characteristics of the relative spectral sensitivity of the optical radiation receiver used in the measurement and the water absorption spectrum in the first, second, and third wavelength ranges, respectively.

10. A method according to claim 9, wherein the described coefficients, obtained by processing the known characteristics of the relative spectral sensitivity of the optical radiation receiver used in the measurement and the water absorption spectrum in the first, second, and third wavelength ranges, are calculated in advance according to the following expressions: [symbol] ₁₃= [symbol] ₃S₃/ [symbol] ₁/S₁ and [symbol] ₂₃= [symbol] ₃S₃/ [symbol] ₂/S₂, where [symbol] ₁, [symbol] ₂, and [symbol] ₃ are the average values of the water absorption coefficients in the first, second, and third wavelength ranges, respectively; S₁, S₂, and S₃ are the average values of the relative spectral sensitivity of the optical radiation receiver in the first, second, and third wavelength ranges, respectively.
